(19) United States
(12) Patent Application Publication
(10) Pub. No.: US 2014/0348702 A1
(43) Pub. Date: Nov. 27, 2014

(54) Method of Controlling the Generation of a Gas Within a Package

(71) Applicant: Cryovac, Inc., Duncan, SC (US)

(72) Inventors: Joshua D. Wofford, Simpsonville, SC (US); Patrick Wayne Thompson, Simpsonville, SC (US); Rick Merical, Mooresburg, TN (US); Russell F. Darley, Simpsonville, SC (US); Nicholas James Berendt, Bournemouth (UK); Jerry Christopher Richardson, Simpsonville, SC (US); Anton L. Timmons, Mauldin, SC (US); Garry William Crossdale, Derbeyshire (UK); Kenneth J. Roach, Mount Pleasant, WI (US)

(73) Assignee: Cryovac, Inc., Duncan, SC (US)

(21) Appl. No.: 14/230,018

(22) Filed: Mar. 31, 2014

Related U.S. Application Data
(60) Provisional application No. 61/825,588, filed on May 21, 2013.

(51) Int. Cl. A61L 2/20

(52) U.S. Cl. CPC A61L 2/20
USPC 422/29; 422/186.3; 422/129

(57) ABSTRACT

The presently disclosed subject matter relates generally to a system and method of generating at least one disinfecting gas within a package through a photochemical reaction. More particularly, the presently disclosed subject matter is directed to a system and method for controlled release of a generated gas from a polymeric film or lamination through selection of a suitable combination of lighting conditions.

### CROSS REFERENCE TO RELATED APPLICATIONS

This application claims the benefit of U.S. Provisional Patent Application No. 61/825,588, filed May 21, 2013, which is incorporated by reference herein in its entirety.

### FIELD OF THE INVENTION

The presently disclosed subject matter relates generally to a system and method for the sustained controlled release of at least one gas within a package. More particularly, the presently disclosed subject matter is directed to a system and method for the controlled release of a sterilizing, sanitizing, and/or disinfecting gas through the selection and maintenance of a photochemical reaction.

### BACKGROUND

In the fields of medicine, pharmaceuticals, and food processing there is a critical need for sterilization to protect against the danger of harmful microorganisms. Current industry practices typically employ the use of steam autoclaving to sterilize a wide range of objects. Particularly, steam autoclaving exposes an object to steam at a temperature of about 121° C. at 15-20 lbs per square inch of pressure for 15-30 minutes. The heat and pressure penetrate the object being sterilized and after a sufficient time kill the harmful microorganisms. However, steam autoclaving requires significant user space and resources. Specifically, autoclaving requires cumbersome equipment, a power supply, and extensive plumbing. In addition, steam autoclaving is not suitable for many plastics and other heat sensitive materials.

Gamma irradiation is also commonly used in the sterilization of medical devices and the like. Specifically, gamma irradiation involves the exposure of an item to a radioactive Cobalt-60 source for a defined amount of time to generate free radicals and other activated molecules that damage the biological components of contaminating bacteria, fungi, and viruses, thereby ensuring their inactivation. The time and exposure to the Cobalt-60 source is used to calculate a total radiation dose for a treated package or pallet, typically ranging from about 50-100 kGy. The required dose for each application is determined based on density, mass, materials, packaging, loading, and the like. However, the free radicals and activated molecules generated as a result of exposure to gamma irradiation have been shown to compromise the mechanical properties of polymers.

Alternatively, sterilant gases (e.g., chlorine dioxide, hydrogen peroxide, and ethylene oxide) can be used to kill or control the growth of microbial contaminants. Chlorine dioxide, for example, is a powerful biocide that can kill fungus, bacteria, and viruses at levels of 0.1 to 15,000 parts per million in contact times of 10 minutes or less. However, one drawback with many of the sterilant gases is that they typically can be used only in limited concentrations and require special handling. In addition, certain sterilants (such as chlorine dioxide, ozone and hydrogen peroxide) are difficult, unsafe, and expensive to transport. Further, many of the sterilant gases must be generated at or near the point of use through on-site plants, which are costly and require significant space to implement.

Packages are commonly sterilized using large scale batch sterilization methods (i.e., large scale ethylene oxide chamber sterilization). Such large scale sterilization methods are costly since there must be enough working capital to create enough inventory to fill a sterilization chamber, ship the inventory to the sterilizer, and execute the sterilization cycle. It would therefore be much more cost effective to sterilize individual packages by constructing one package, one product, and using one complete sterilization cycle.

It is known in the art to generate sterilant gases in individual packages via light activation. Specifically, packages comprising a photocatalyst and anions capable of being oxidized by the activated catalyst or a byproduct thereof are known in the art and can be activated through exposure to a light source. However, these packages are limited to a short, initial activation of gas production as opposed to prolonged or sustained generation. Such a short burst of sterilizing gas can be subject to decomposition or breakdown before sterilization is complete.

Thus, there is a need for simple and inexpensive methods and devices for sterilizing objects in a safe and efficient manner. Particularly, it would be desirable to provide a system and method that provides for the sustained generation of a gas in individual packages over a desired time course (i.e., a high concentration of sterilizing gas maintained for an extended period of time).

### SUMMARY

In some embodiments, the presently disclosed subject matter is directed to a system comprising a first area, a second area, and a package. Particularly, the first area comprises at least one light source with a wavelength of about 350 to 1000 nm and a relative intensity of about 100. The second area comprises at least one light source with a wavelength of about 385 to 750 nm and a relative intensity of 0.001 to 50. The package comprises a composition that generates a disinfecting gas upon exposure to the first and second light sources and an object to be disinfected.

In some embodiments, the presently disclosed subject matter is directed to a method of controlling the generation of a disinfecting gas within a package interior. Specifically, the method comprises providing an unactivated package comprising a composition that generates a disinfecting gas upon exposure to first and second light sources and an object to be disinfected. The method further comprises introducing the package to a system comprising a first area and a second area that include light sources. The system exposes the package to at least one light source with a wavelength of 350 to 1000 nm and a relative intensity of about 100 for a period of about 1 to 3 minutes followed by immediate exposure to at least one light source with a wavelength of 385 to 750 nm and a relative intensity of about 0.001 to 50 for a period of about 10 to 30 minutes such that the package exhibits a controlled generation of the gas.

In some embodiments, the presently disclosed subject matter is directed to a method of providing sustained generation of a disinfecting gas within a package. The method comprises providing an unactivated package comprising a composition that generates a disinfecting gas upon exposure to first and second light sources and an object to be disinfected. The method further comprises introducing the package to a system comprising a first area and a second area that include light sources. The system exposes the package to at least one light source with a wavelength of 350 to 1000 nm and a relative intensity of about 100 for a period of about 1 to 3 minutes followed by immediate exposure to at least one light source with a wavelength of 385 to 750 nm and a relative intensity of about 0.001 to 50 for a period of about 10 to 30 minutes such that the package exhibits sustained generation of the gas at a level of 400 ppmv or less.

In some embodiments, the presently disclosed subject matter is directed to a system comprising an area comprising at least one light source comprising first and second sets of lenses, mirrors, filters, or combinations thereof. The first set provides a wavelength of about 350 to 1000 nm and a relative intensity of about 100 and the second set provides a wavelength of about 385 to 750 nm and a relative intensity of 0.001 to 50. The system further comprises a package comprising a composition that generates a disinfecting gas upon exposure to the light source and an object to be disinfected.

In some embodiments, the presently disclosed subject matter is directed to a method of controlling the generation of a disinfecting gas within a package interior. Specifically, the method comprises providing an unactivated package comprising a composition that generates a disinfecting gas upon exposure to the light source and an object to be disinfected. The package is then introduced to a system comprising an area comprising at least one light source comprising first and second sets of lenses, wherein the system exposes the package to a wavelength of 350 to 1000 nm and a relative intensity of about 100 for a period of about 1 to 3 minutes followed by immediate exposure to a wavelength of 385 to 750 nm and a relative intensity of about 0.001 to 50 for a period of about 10 to 30 minutes, such that the package exhibits a controlled generation of the gas.

In some embodiments, the presently disclosed subject matter is directed to a method of providing sustained generation of a disinfecting gas within a package. The method comprises providing an unactivated package comprising a composition that generates a disinfecting gas upon exposure to the light source and an object to be disinfected. The package is then introduced to a system comprising an area comprising at least one light source comprising first and second sets of lenses, wherein the system exposes the package to a wavelength of 350 to 1000 nm and a relative intensity of about 100 for a period of about 1 to 3 minutes followed by immediate exposure to a wavelength of 385 to 750 nm and a relative intensity of about 0.001 to 50 for a period of about 10 to 30 minutes, such that the package exhibits sustained generation of the gas at a level of 400 ppmv or less.

### BRIEF DESCRIPTION OF THE DRAWINGS

FIG. 1 is a chlorine dioxide release profile after exposing a disclosed package to halogen light.

FIG. 2 is a chlorine dioxide release profile after exposing a disclosed package to fluorescent light.

FIG. 3 is a chlorine dioxide release profiles after exposing a disclosed package to one fluorescent bulb versus two fluorescent bulbs.

FIG. 4 is a chlorine dioxide release profile after exposing a disclosed package to halogen light, followed by exposure to fluorescent light.

FIG. 5 is a chlorine dioxide release profile after exposing a disclosed package to halogen light, followed by exposure to LED light.

### DETAILED DESCRIPTION

### I. General Considerations

The presently disclosed subject matter is generally directed to a system of generating a gas within a package by means of a photochemical reaction. More particularly, a composition capable of generating a gas upon exposure to light is incorporated into the disclosed package. As set forth in detail herein below, the package is introduced to a system comprising a first area comprising at least one high intensity light source that functions to rapidly trigger the production of a disinfecting gas within the package headspace. The system further comprises a second area comprising at least one lower intensity light source (compared to the intensity of the first area light source) that functions to maintain the disinfecting gas production within the package at a stable level for a desired period of time.

As set forth in more detail herein below, the selection of light sources allows a rapid increase in the generation of sterilant gas followed by maintenance of the gas for an extended period of time. For example, use of a halogen bulb can increase the gas concentration very quickly due to its intensity and wavelength. However, after the initial activation, it will decompose the disinfecting gas readily because of its peak at 365 nm. Advantageously, the disclosed system and methods employ a secondary light source that does not include a strong peak at 365 nm (such as an LED light, for example) to allow for sustained generation of the gas.

Although described herein with respect to first and second light sources, the disclosed system can comprise any number of light sources (i.e., one or more light sources). Thus, the disclosed package is introduced to a system where it is exposed to a combination of light sources of sufficient intensity and for a residence time satisfactory to rapidly activate the production of a gas and to maintain its production over a desired time course.

### II. Definitions

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs.

Following long standing patent law convention, the terms “a”, “an”, and “the” refer to “one or more” when used in the subject application, including the claims. Thus, for example, reference to “a package” includes a plurality of such packages, and so forth.

Unless indicated otherwise, all numbers expressing quantities of components, reaction conditions, and so forth used in the specification and claims are to be understood as being modified in all instances by the term “about.” Accordingly, unless indicated to the contrary, the numerical parameters set forth in the instant specification and attached claims are approximations that can vary depending upon the desired properties sought to be obtained by the presently disclosed subject matter.

As used herein, the term “about”, when referring to a value or to an amount of mass, weight, time, volume, concentration, percentage, and the like can encompass variations of, and in some embodiments, ±20%, in some embodiments ±10%, in some embodiments ±5%, in some embodiments ±1%, in some embodiments ±0.5%, and in some embodiments ±0.1%, from the specified amount, as such variations are appropriated in the disclosed films and methods.

The term “disinfecting” or “disinfected” refers to the process of cleansing to destroy and prevent the growth of pathogenic microorganisms. In some embodiments, disinfecting can include a combination of a concentration of disinfecting gas and a time exposure interval that will disinfect an object subjected to the gas within a package. Disinfecting conditions can be provided by a wide range of disinfecting gas concentrations in combination with various time intervals. In general, the higher the concentration of a disinfecting gas, the shorter a corresponding time interval is needed to establish disinfecting conditions. Accordingly, the effective amount of a disinfecting gas can vary depending upon the length of exposure of the object to the gas. Further, although disinfecting conditions are described herein, in some embodiments the presently disclosed subject matter can equally be used to sterilize a product. Thus, when the term “disinfecting” is used, there are some embodiments where the term includes both disinfecting and sterilizing.

As used herein, the term “disinfecting gas” refers to a gas that effectively destroys, neutralizes, and/or inhibits the growth of pathogenic microorganisms without adversely affecting the object being disinfected. In some embodiments, disinfecting gas includes (but is not limited to) chlorine dioxide, ethylene oxide, sulfur dioxide, nitrogen dioxide, nitric oxide, nitrous oxide, carbon dioxide, hydrogen sulfide, hydrocyanic acid, dichlorine monoxide, ozone, vaporous hydrogen peroxide, and the like. However, this list is not exhaustive and disinfecting gases suitable for use with the presently disclosed subject matter can include any gas that is capable of disinfecting a product.

As used herein, the term “film” is not limiting and refers to a variety of different flexible materials that can be used in conjunction with the disclosed system including without limitation: a laminate, a web, a sheet, a bag, a pouch, a coating on a substrate or carrier, and the like.

The term “first area” or “second area” as used herein refers to any enclosure with a light source where light-induced gas generation can be carried out.

The term “immediately” as used herein refers to any time point falling between the removal of the disclosed package from exposure to a first light source (e.g., a high intensity light) until the time of exposure to a second light source (e.g., medium to low intensity light). For example, in some embodiments the term “immediately” can include 5 seconds or less, 10 seconds or less, 30 seconds or less, 1 minute or less, 3 minutes or less, 5 minutes or less, or 10 minutes or less. It should also be appreciated that greater or less amounts of time are also intended to be included within the term “immediately.”

The term “interior” as used herein with regard to a package refers to the actual inside portion of the package into which an object is inserted.

The term “light source” as used herein encompasses all light generating elements and types, including (but not limited to) incandescent, fluorescent, discharge, ultraviolet, glow, tungsten halogen, xenon, metal halide, high pressure mercury vapor, arc lamps, sodium vapor lamps, high pressure sodium lamps, induction lamps, electroluminescent, OLED, LED, and the like. The term “light source” as used herein can further refer to a single light source, as well as to a plurality of light sources.

The term “medical product” as used herein refers to any product that is sterilized and/or sanitized prior to use in health care, whether for medical, dental, or veterinary applications. Such products can include (but are not limited to) needles, syringes, sutures, bandages, general wound dressings, non-adherent dressings, burn dressings, surgical tools (such as scalpels, gloves, drapes, and other disposal items), surgical implants, surgical sutures, stents, catheters, vascular grafts, artificial organs, cannulas, wound care devices, dialysis shunts, wound drain tubes, skin sutures, vascular grafts, implantable meshes, intraocular devices, heart valves, biological graft materials, tape closures and dressings, head coverings, shoe coverings, sterilization wraps, and the like. Thus, the term “medical product” can include any instrument, apparatus, implement, machine, appliance, contrivance, implant, or other similar or related article, including any component or part that is intended for use in the cure, mitigation, treatment, or prevention of disease, or intended to affect the structure or any function of the body of a human or animal.

As used herein, the term “object” refers to the article or material being acted upon to be sterilized, sanitized, disinfected, and/or decontaminated by the disclosed methods and systems. The term can include any material to be sterilized, disinfected, and/or decontaminated, no matter the physical form. Thus, an object can include, for example, a medical device, medical instrument, or any other article or combination of articles for which disinfection is desired. An object in accordance with the presently disclosed subject matter can have a wide variety of shapes and sizes and can be made from a variety of materials (e.g., without limitation, metal, plastic, glass, and the like).

As used herein, the term “package” refers to the combination of all of the various components used in the packaging of an object. The package can in some embodiments be inclusive of, for example, a rigid support member, all films used to surround the object and/or the rigid support member, an absorbent component, and even the atmosphere within the package, together with any additional components used in the packaging of the object acting as a container. In some embodiments, the term “package” can also include any of the wide variety of containers known in the art.

The term “relative intensity” refers to the ratio of the intensity of the target lamp divided by the intensity of the high intensity lamp. For example, in some embodiments the high intensity light source can be halogen or LED light. Relative light intensity also references a specific wavelength or wavelength range over which the two lamps are compared. As can be appreciated by those of ordinary skill in the art, the relative intensity is normalized to 100, such that the high intensity lamp is always 100.

The term “sterilization” as used herein refers to the elimination or destruction of all living microorganisms within a defined system. In the case of a sealed package, for example, the system is defined as the area enclosed by the sealed packaging materials (i.e., the packaged article/device and the headspace).

All compositional percentages used herein are presented on a “by weight” basis, unless designated otherwise.

### III. The Disclosed Package

III.A. Generally

As set forth above, the presently disclosed subject matter relates generally to a system and method for the activation of a composition by a combination of light sources to provide controlled sustained generation and release of at least one gas. Specifically, a gas-generating composition is incorporated within a package and the package is exposed to the system disclosed herein to generate a desired gas within the package headspace.

III.B. The Disclosed Composition

The disclosed gas-generating composition includes an energy-activated catalyst and anions capable of being oxidized by the activated catalyst surface or a subsequent reaction product to generate a gas. To this end, the disclosed composition comprises from about 30 to 99.9 weight percent energy-activated catalyst, in some embodiments, about 50 to 99 weight percent energy-activated catalyst; in some embodiments, from about 80 to 98 weight percent energy-activated catalyst; and in some embodiments, from about 86 to 96 weight percent energy-activated catalyst. Thus, the presently disclosed subject matter includes embodiments wherein the composition comprises at least about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 99.9 weight percent energy-activated catalyst.

Any semiconductor activated by electromagnetic energy, or a particle or other material incorporating such a semiconductor, can be used as the energy-activated catalyst of the composition. Such semiconductors can be metallic, ceramic, inorganic, or polymeric materials prepared by various processes known in the art, such as sintering. In some embodiments, the semiconductors can be surface treated or encapsulated with materials such as silica or alumina to improve durability, dispersibility or other characteristics of the semiconductor. Catalysts for use in the presently disclosed subject matter are commercially available in a wide range of particle sizes from nanoparticles to granules.

In some embodiments, representative energy-activated catalysts can include (but are not limited to) metal oxides (such as anatase, rutile or amorphous titanium dioxide, zinc oxide, tungsten trioxide, ruthenium dioxide, iridium dioxide, tin dioxide, strontium titanate, barium titanate, tantalum oxide, calcium titanate, iron (Ill) oxide, molybdenum trioxide, niobium pentoxide, indium trioxide, cadmium oxide, hafnium oxide, zirconium oxide, manganese dioxide, copper oxide, vanadium pentoxide, chromium trioxide, yttrium trioxide, silver oxide, and/or TiₓZr₁₋ₓO₂, wherein x is between 0 and 1). In some embodiments, suitable catalysts can include (but are not limited to) metal sulfides, such as cadmium sulfide, zinc sulfide, indium sulfide, copper sulfide, tungsten disulfide, bismuth trisulfide, or zinc cadmium disulfide. In some embodiments, presently disclosed catalysts can include (but are not limited to) metal chalcogenites (such as zinc selenide, cadmium selenide, indium selenide, tungsten selenide, or cadmium telluride), metal phosphides (such as indium phosphide), metal arsenides (such as gallium arsenide), nonmetallic semiconductors (such as silicon, silicon carbide, diamond, germanium, germanium dioxide, and germanium telluride), photoactive homopolyanions (such as W₁₀O₃₂⁻⁴), photoactive heteropolyions (such as XM₁₂O₄₀⁻ⁿ or X₂M₁₈O₆₂⁻⁷, wherein x is Bi, Si, Ge, P or As, M is Mo or W, and n is an integer from 1 to 12), and/or polymeric semiconductors (such as polyacetylene).

The disclosed composition comprises from about 0.01 to 50 weight percent anions; in some embodiments, about 2 to 20 weight percent anions; and in some embodiments, from about 4 to 14 weight percent of a source of anions capable of being oxidized by the activated catalyst or reacted with species generated during activation of the catalyst to generate a gas. Therefore, the disclosed composition can comprise at least about 0.01, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 weight percent of a source of anions capable of being oxidized by the activated catalyst or reacted with species generated during activation of the catalyst to generate a gas.

Any source containing anions capable of being oxidized by the activated catalyst or reacted with species generated during excitation of the catalyst to generate a gas can be used in the disclosed composition. An anion is capable of being oxidized by the activated catalyst to generate a gas if its oxidation potential is such that it will transfer an electron to a valence band hole of the energy-activated catalyst. In some embodiments, suitable solids containing anions can include a salt of the anion and a counterion; an inert material such as a sulfate, a zeolite, or a clay impregnated with the anions; a polyelectrolyte such as polyethylene glycol, an ethylene oxide copolymer, or a surfactant; a solid electrolyte or ionomer such as nylon or Nafion™ (DuPont Dow Elastomers (Wilmington, Del., United States of America)); or a solid solution. When the composition is a solids-containing suspension, a salt dissociates in a solvent to form a solution including anions and counterions, and the energy-activated catalyst is suspended in the solution. A powder can be formed, for example, by drying this suspension or by physically blending the solid (e.g., salt particles) with the energy-activated catalyst particles.

Salts suitable for use as the anion source can include (but are not limited to) an alkali metal chlorite, an alkaline-earth metal chlorite, a chlorite salt of a transition metal ion, a protonated primary, secondary or tertiary amine, or a quaternary amine, an alkali metal bisulfite, an alkaline-earth metal bisulfite, a bisulfite salt of a transition metal ion, a protonated primary, secondary or tertiary amine, or a quaternary amine, an alkali metal sulfite, an alkaline-earth metal sulfite, a sulfite salt of a transition metal ion, a protonated primary, secondary or tertiary amine, or a quaternary amine, an alkali metal sulfide, an alkaline-earth metal sulfide, a sulfide salt of a transition metal ion, a protonated primary, secondary or tertiary amine, or a quaternary amine, an alkali metal bicarbonate, an alkaline-earth metal bicarbonate, a bicarbonate salt of a transition metal ion, a protonated primary, secondary or tertiary amine, or a quaternary amine, an alkali metal carbonate, an alkaline-earth metal carbonate, a carbonate salt of a transition metal ion, a protonated primary, secondary or tertiary amine, or a quaternary amine, an alkali metal hydrosulfide, an alkaline-earth metal hydrosulfide, a hydrosulfide salt of a transition metal ion, a protonated primary, secondary or tertiary amine, or a quaternary amine, an alkali metal nitrite, an alkaline-earth metal nitrite, a nitrite salt of a transition metal ion, a protonated primary, secondary or tertiary amine, or a quaternary amine, an alkali metal hypochlorite, an alkaline-earth metal hypochlorite, a hypochlorite salt of a transition metal ion, a protonated primary, secondary or tertiary amine, or a quaternary amine, an alkali metal cyanide, an alkaline-earth metal cyanide, a cyanide salt of a transition metal ion, a protonated primary, secondary or tertiary amine, or a quaternary amine, an alkali metal peroxide, an alkaline-earth metal peroxide, or a peroxide salt of a transition metal ion, a protonated primary, secondary or tertiary amine, or a quaternary amine. In some embodiments, preferred salts can include sodium, potassium, calcium, lithium or ammonium salts of a chlorite, bisulfite, sulfite, sulfide, hydrosulfide, bicarbonate, carbonate, hypochlorite, nitrite, cyanide or peroxide. Commercially available forms of chlorite and other salts suitable for use can contain additional salts and additives such as tin compounds to catalyze conversion to a gas.

The disinfecting gas released by the disclosed composition will depend upon the anions that are oxidized or reacted. Any gas formed by the loss of an electron from an anion, by reaction of an anion with electromagnetic energy-generated protic species, by reduction of a cation in an oxidation/reduction reaction, or by reaction of an anion with a chemisorbed molecular oxygen, oxide or hydroxyl radical can be generated and released by the composition. For example, in some embodiments, the gas can be chlorine dioxide, sulfur dioxide, hydrogen sulfide, hydrocyanic acid, nitrogen dioxide, nitric oxide, nitrous oxide, carbon dioxide, dichlorine monoxide, chlorine, or ozone.

In some instances, the disclosed composition can include two or more different anions to release two or more different gases at different rates. The gases can be released for different purposes or so that one gas will enhance the effect of the other gas. For example, a composition containing bisulfite and chlorite anions can release sulfur dioxide for food preservation and chlorine dioxide for control of microorganisms.

The disclosed composition can be a solid (such as a powder), film, tablet, coating, or a liquid (e.g., dispersion, emulsion), depending upon its intended use. For example, when a powder is exposed to electromagnetic energy, the energy-activated catalyst is activated, the anions are oxidized or reacted with species generated during excitation of the catalyst to generate the gas, and the gas diffuses through the powder and is released.

The disclosed composition can comprise any of a wide variety of additives known in the art. Such additives can include (but are not limited to) colorants, dyes, fragrances, fillers, lubricants, stabilizers, accelerators, retarders, enhancers, blending facilitators, controlled release agents, antioxidants, UV blockers, mold release agents, plasticizers, biocides, flow agents, anti-caking agents, processing aids, and/or light filtering agents. Continuing, additives such as UV blockers can also be included in the disclosed composition if it is desirable to limit the wavelength range transmitted to the energy-activated catalyst. Photosensitizers can further be added to shift the absorption wavelength of the composition, particularly to shift an ultraviolet absorption wavelength to a visible absorption wavelength to improve activation by room lighting. UV absorbers can be added to the composition to slow the gas generation and release rate.

III.C. The Disclosed Package

Applications for the disclosed gas-generating composition are numerous. For example, the composition can be incorporated into a package for use in disinfecting the package contents. Particularly, the composition can be impregnated, melt processed, sintered, blended with other powders, or otherwise incorporated into a variety of materials to provide films, fibers, coatings, tablets, resins, polymers, plastics, tubing, membranes, engineered materials, paints and adhesives for a wide range of end use applications. For example, the disclosed composition can in some embodiments be incorporated into injection-molded, compression-molded, thermal-formed and/or extrusion-formed polymeric products by compounding and/or pelletizing the disclosed composition via conventional means and mixing the pellets with a material before the conventional forming or molding process. Thus, in some embodiments, the composition can be useful in preparing a wide variety of injection-molded products, compression-molded products, thermal-formed products, and/or extrusion-formed products, such as cast or blown films.

For instance, a multilayered package can be formed to generate a gas within the package interior. In such embodiments, the package can include a gas generating layer comprising the disclosed composition. Particularly, the gas-generating layer can comprise an energy-activated catalyst capable of being activated by electromagnetic energy and anions capable of being oxidized or reacted to generate a gas. In some embodiments, the disclosed package can comprise a barrier layer positioned adjacent to a surface of the gas generating layer. The barrier layer can in some embodiments be transparent to electromagnetic energy such that it transmits the energy to the gas generating layer. The barrier layer can be impermeable or only semipermeable to the gases generated and released by the gas generating layer.

Therefore, the surface of a material (such as a polymeric film) or the entire material itself can be impregnated or coated with the disclosed composition. Alternatively or in addition, the composition can be admixed with a material, the composition can be enclosed within a gas-permeable container, and/or the material and the composition can be enclosed within a container. When the composition is enclosed within a container, the container can be hermetically sealed using methods well known in the packaging art.

Products (such as packages, films, and the like) can include between about 0.1 and 70 weight percent of the disclosed composition; in some embodiments, from about 1 to 50 weight percent of the disclosed composition; and in some embodiments, from about 2 to 50 weight percent of the disclosed composition. Thus, the composition can be present in a product in an amount of about 0.1, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 weight percent, based on the total weight of the product. As would be appreciated by those of ordinary skill in the art, the remainder of the products can be comprised of polymeric materials, additives, and the like.

III.D. Object

The disclosed package can be used to house any of a wide variety of objects known in the art. For example, in some embodiments, the disclosed package can be used to house medical products, food products, various industrial and consumer products, and other like objects.

### IV. The Disclosed System

It has been surprisingly discovered that controlled sustained release of a gas (such as chlorine dioxide) can be generated within a package comprising the disclosed composition by exposing the package to an initial high level of electromagnetic energy, followed by a reduced exposure to electromagnetic energy. For example, in some embodiments, the disclosed system can include an area housing one or more light sources that the package travels through or across. Particularly, the package can travel to a first area where it is exposed to high intensity light to initiate generation of a gas within the package interior. The package can then travel to a second area where it is exposed to less intense light (compared to the first area) to maintain generation of the gas. A stationary package could also be exposed to a high intensity light for a period of time to initiate production of the gas. After exposure to the high intensity light ceases, the package is exposed to a less intense light to maintain the concentration of gas within the package.

In some embodiments, the described first and second areas can be combined into a single area that enables both high and low intensity light. For example, the single area can comprise one or more light sources with a combination of lenses and/or mirrors to accommodate both high intensity and lower intensity light. Particularly, the lenses and/or mirrors can concentrate a light source to expose a package to an initial high intensity light. After a desired amount of time, the lenses and/or mirrors can be removed or repositioned to expose the package to a less intense light (compared to the initial high intensity light). Alternatively or in addition, in some embodiments, the single area can be configured to include a spiral conveyor such that the first loops of the conveyor are positioned relatively close to the light source (so that the package is exposed to a high intensity light) and the last loops of the conveyor are positioned relatively far from the light source (so that the package is exposed to a less intense light compared to the first few conveyor loops).

Any electromagnetic energy source capable of activating an energy-activated catalyst of the disclosed composition can be included within the presently disclosed subject matter. In other words, any electromagnetic energy source that provides a photon having energy in excess of the band gap of the energy-activated catalyst is suitable. In some embodiments, suitable electromagnetic energy sources can include sunlight, fluorescent light, ultraviolet light, tungsten halogen, LED, xenon, metal halide, high pressure mercury vapor, arc lamps, sodium vapor lamps, high pressure sodium lamps, induction lamps, electroluminescent lamps, OLED, and the like for photo-activation of the disclosed composition.

The first and second areas of the disclosed system can in some embodiments be contained within a housing constructed from any of a wide variety of materials known in the art. The first and second areas can be configured with at least one bulb or lamp of desired intensity. Thus, in some embodiments, the first and second areas house a plurality of bulbs disposed in a lamp bank where each lamp is spaced apart and substantially parallel to another lamp. In other embodiments, the lamps can be arranged in other orientations, such as in non-linear arrangements. In some embodiments, the lamp bank can have from about 1 to 1,000 lamps, although more lamps can be included within the scope of the presently disclosed subject matter. Thus, the lamp bank can have a wide variety of structures and forms, as would be known in the art.

In some embodiments, the first and second areas can optionally include an entrance path and an exit path such that a package can continuously move into and out of each area after a desired amount of exposure time. In some embodiments, the areas can be configured with at least one curved portion or segment to ensure even and consistent exposure to the light source. In addition, a circular or spiral configuration allows light exposure to contact all sides of the package during treatment. As set forth herein above, the distance of the package path to the light source can be varied (i.e., the first several loops of a spiral conveyor can be relatively close to a light source and the last loops can be relatively removed from a light source). In some embodiments, the first and second areas are completely enclosed such that no light energy can escape unused and to ensure that human eyes and skin are prevented from becoming exposed to the light. One of ordinary skill in the art would recognize that the disclosed first and second areas are not limited and can be constructed in any of a wide variety of shapes known in the art and that all features disclosed herein are optional.

A package comprising the disclosed composition passes initially into a first area comprising one or more sources of high intensity light, such as sunlight, fluorescent light, ultraviolet light, tungsten halogen, LED, xenon, metal halide, high pressure mercury vapor, arc lamps, sodium vapor lamps, high pressure sodium lamps, induction lamps, electroluminescent lamps, OLED, or combinations thereof. The high intensity light sources expose the package to an initial wavelength of about 300 to 1000 nm; in some embodiments, about 310 to 850 nm; in some embodiments, about 330 to 700 nm; and in some embodiments, about 350 to 600 nm. Thus, the package is initially exposed to a high intensity light in the first area at a wavelength of about 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nm.

In addition to wavelength, light intensity is also a critical factor for activation of gas production in the disclosed package. Halogen and LED light sources are examples of high intensity light sources in the optimum activation wavelengths of 350 to 500 nm. CFL or fluorescent-type bulbs have a lower intensity in the optimum activation wavelength window (i.e., 0-50% of the intensity of the high intensity sources).

The package can be exposed to the high intensity light source for a time of about 30 seconds to about 7 minutes; in some embodiments, about 45 seconds to about 5 minutes; and in some embodiments, about 1 minute to about 3 minutes. Thus, the package can be exposed to high intensity light for a period of about 0.5, 0.75, 1, 1.25, 1.5, 1.75, 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, or 7 minutes. The high intensity light triggers an initial rapid activation of gas production within the package interior.

After a desired amount of time, the package passes into a second area that exposes the package to a medium or low intensity light source to maintain gas production within the package interior. To this end, the second area comprises one or more sources of medium and/or low intensity light, such as sunlight, fluorescent light, ultraviolet light, tungsten halogen, LED, xenon, metal halide, high pressure mercury vapor, arc lamps, sodium vapor lamps, high pressure sodium lamps, induction lamps, electroluminescent lamps, OLED, and the like. The light sources in the second area expose the package to a wavelength of about 350 to 725 nm; in some embodiments, about 360 to 600 nm; and in some embodiments, about 385 to 500 nm. Thus, the package is exposed to a medium to low intensity light in the second area at a wavelength of about 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, or 725 nm.

The package can be exposed to the medium or low intensity light source in the second area for about 5 minutes to 45 minutes; in some embodiments, about 7 minutes to about 40 minutes; in some embodiments, about 8 minutes to about 35 minutes; and in some embodiments, about 10 minutes to about 30 minutes. Thus, the package can be exposed to low or medium intensity light in the second area for a period of about 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5 40 40.5, 41, 41.5, 42, 42.5, 43, 43.5, 44, 44.5, or 45 minutes.

Although the disclosed system describes first and second areas that each include at least one light source, it should be appreciated that the system can include embodiments with a greater or lesser number of areas. Thus, the term “an area” can comprise one or more areas, such that a disclosed package can be exposed to at least one area of high intensity light, followed by at least one area of lower intensity light as described herein.

### V. The Disclosed Method

The presently disclosed subject matter includes a method for the controlled sustained release of a gas to sterilize and/or disinfect objects, such as for medical applications. It has been surprisingly discovered that when a package comprising the disclosed composition is initially exposed to a high intensity light source for a relatively short period of time (such as about 3 to 5 minutes), the composition rapidly generates a disinfecting gas (such as chlorine dioxide). In addition, it has further been discovered that when the package is thereafter exposed to a medium or low intensity light source for a relatively longer period of time (such as about 10 to 30 minutes), the gas generation is maintained at a high concentration.

In some embodiments, the level of gas generated during the initial exposure to the high intensity light source can be about 400 ppmv. The level of gas production is decreased during the second phase of exposure (during exposure to the medium or low intensity light source). Therefore, the first phase of the system (exposure to the high intensity light source) is characterized by a high rate of gas production, resulting in an increased concentration of gas within the package. The second phase is characterized by a lower rate of gas production to maintain the gas concentration obtained in the first phase. The concentration of gas is maintained at about the same level throughout the second phase (i.e., no significant increase or decrease in gas concentration).

As set forth herein above, the disclosed composition generates a desired gas when exposed to electromagnetic energy. Particularly, it is believed that the energy-activated catalyst of the composition absorbs a photon having energy in excess of the band gap and an electron is promoted from the valence band to the conduction band, producing a valence band hole. The valence band hole and electron diffuse to the surface of the energy-activated catalyst where each can chemically react. An anion is oxidized by the activated catalyst surface when an electron is transferred from the anion to a valence band hole, forming the gas. It is believed that chlorine dioxide or nitrogen dioxide is generated by the transfer of an electron from a chlorite or nitrite anion to a valance band hole. These and other gases, such as ozone, chlorine, carbon dioxide, nitric oxide, sulfur dioxide, nitrous oxide, hydrogen sulfide, hydrocyanic acid, and dichlorine monoxide, can also be formed via reaction of an anion with protic species generated during activation of the catalyst by abstraction of an electron from water, chemisorbed hydroxyl, or some other hydrated species. See, U.S. Pat. No. 7,273,567, the entire disclosure of which is hereby incorporated by reference.

The disinfecting gas diffuses out of the disclosed composition into the surrounding atmosphere (such as the interior of a package) for a period of up to about 24 hours. In some embodiments, the gas can be used to retard, control, kill and/or prevent microbiological contamination (e.g., bacteria, fungi, viruses, mold spores, algae, and protozoa). Alternatively or in addition, the gas can be used to deodorize, enhance freshness, and/or retard, prevent, inhibit, and/or control chemotaxis.

As would be known to those of skill in the art, the rate of gas release from the disclosed composition, activation of the composition to initiate gas release, and the release rate profile can be altered in various ways. For example, the concentration of energy-activated catalyst or anion source in the disclosed composition can be changed. Alternatively or in addition, a base, surfactant, diluent, or light filtering additive can be added to the composition. Further, materials (such as silicates) can be added to complex active surface sites. Continuing, charge, lattice, or surface defects can be introduced in the catalyst (e.g., Ti⁽⁺³⁾ impurities in titanium-based catalysts). Further, the method of processing the composition, light wavelength and intensity, and/or the order of addition of ingredients in preparing the composition can all be changed. Such modifications are well known to those of ordinary skill in the art.

It should be recognized that the methods listed herein above are not intended to be limiting. Rather, the presently disclosed subject matter includes the methods listed herein above, in addition to any of a wide variety of methods known in the art.

### VI. Advantages of the Presently Disclosed Subject Matter

The presently disclosed subject matter is directed to improved methods of generating a desired gas within a package. Particularly, the disclosed method optimizes the conditions needed to activate gas production and maintain production at an optimal level, thereby conserving resources.

Further, by optimizing the conditions needed to retard, prevent or control biological contamination, disinfection time is reduced, thereby allowing more efficient time to market.

The disclosed system provides for the sterilization of individual packages as opposed to the large scale sterilization methods known in the art. Individual package sterilization is much safer than large scale sterilization and avoids user exposure to the explosive nature of some gases (such as ethylene oxide) as well as disposal problems associated with some byproducts (such as cobalt).

In addition, the ability to maintain a sterilizing gas concentration at a desired level for between about 2 and about 30 minutes facilitates a more controlled and robust microbial inactivation or sterilization system.

Although several advantages of the disclosed system are set forth in detail herein, the list is by no means limiting. Particularly, one of ordinary skill in the art would recognize that there can be several advantages to the disclosed system and methods that are not included herein.

### EXAMPLES

The following Examples provide illustrative embodiments. In light of the present disclosure and the general level of skill in the art, those of ordinary skill in the art will appreciate that the following Examples are intended to be exemplary only and that numerous changes, modifications, and alterations can be employed without departing from the scope of the presently disclosed subject matter.

Tables 1 and 2 below list resin identification and film construction information, as follows:

TABLE I
Resin Identification

| Material | ID | Source |
|---|---|---|
| A | Escrorene ® | ExxonMobil (Fairfax, Virginia, United |
| | Ultra LD | States of America) |
| | 761.36 | |
| B | CLARA | Southwest Research Institute (San |
| | | Antonia, Texas, United States of |
| | | America) |
| C | Adcote ® 503E | Rohm & Haas Company (Philadelphia, |
| | | Pennsylvania, United States of |
| | | America) |
| D | Catalyst F | Rohm & Haas Company (Philadelphia, |
| | | Pennsylvania, United States of |
| | | America) |
| E | Mylar ® 822 | DuPont Dow Elastomers (Wilmington, |
| | | Delaware, United States of America) |
| F | LDPE 611A | Dow Chemical Company (Midland, |
| | | Michigan, United States of America) |
| G | BYNEL ® 4157 | E. I. DuPont de Nemours and Company |
| | | (Wilmington, Delaware, United States |
| | | of America) |
| H | Ultramid ® | BASF Corporation (Florham Park, New |
| | B36 01 | Jersey, United States of America) |
| I | SELAR ® PA3426 | E. I. DuPont de Nemours and Company |
| | | (Wilmington, Delaware, United States |
| | | of America) |
| J | EVAL ® H171B | EVALCA/Kuraray (Osaka, Japan) |
| K | Novacote ® NC- | Georgia-Pacific Chemicals (Atlanta, |
| | 250/CA350 | Georgia, United States of America) |
| L | CLEAR BVLC- | National Plastic Co, Ltd. (Seoul, |
| | hxx color code | Korea) |
| | 0T1 | |
| M | Dowlex ® 2070G | Dow Chemical Company (Midland, |
| | | Michigan, United States of America) |

A is an ethylene vinyl acetate copolymer with 26.7% vinyl acetate content, vicat softening temperature of 115° F. (ASTM D1525), tensile strength at break of 1170 psi (ASTM D638), and tensile elongation at break of >800% (ASTM D638).

B is a gas-generating composition in LDPE carrier masterbatch resin (50%/50%), as described in Section III.B.

C is solvent based polyurethane.

D is solvent based polyurethane.

E is biaxially oriented polyester, chemically primed on one side.

F is low density polyethylene with flow rate of 0.70-1.06 g/10 min, density of 0.923-0.925 g/cc, and vicat softening point of 107° C.

G is maleic anhydride modified linear low density polyethylene.

H is polyamide-6 with density of 1.15 g/cc and melting point of 215-225° C.

I is amorphous polyamide.

J is hydrolyzed ethylene/vinyl acetate copolymer (30-40 mol % ethylene) with a melt index of 1.3-2.1 g/10 minutes.

K is a two component isocyanate polyester urethane adhesive.

L is polyvinyl chloride.

M is linear low density ethylene/octene copolymer.

TABLE 2
Film Identification

| Film ID | Layer | Formulation | | Volume % | Mils |
|---|---|---|---|---|---|
| 1 | 1 | 80% | A | 76.92 | 2.0 |
| | | 20% | B | | |
| | 2 | 95% | C | 3.85 | 0.1 |
| | | 5% | D | | |
| | 3 | 100% | E | 19.23 | 0.5 |
| 2 | 1 | 80% | M | 2.8 | 0.41 |
| | | 20% | F | | |
| | 2 | 100% | G | 0.76 | 0.11 |
| | 3 | 80% | H | 0.62 | 0.09 |
| | | 20% | I | | |
| | 4 | 100% | J | 0.83 | 0.12 |
| | 5 | 80% | H | 0.62 | 0.09 |
| | | 20% | I | | |
| | 6 | 100% | G | 0.76 | 0.11 |
| | 7 | 70% | M | 1.79 | 0.26 |
| | | 30% | F | | |
| | 8 | 100% | K | 0.41 | 0.06 |
| | 9 | 100% | L | 91.37 | 13.23 |

### Example 1

### Preparation of Film 1

Layer 1 of Film 1 was prepared on a small scale Leistritz twin screw extruder at 18 g/min. Extrudate was cast onto a chilled drum to produce a 2.0 monolayer film structure. The monolayer film was then pretreated with a corona treater at 1.5 WD to increase surface energy.

The monolayer film (Layer 1 from Table 2) was adhesively laminated to a 48 gauge chemically primed PET sheet (Layer 3 from Table 2). The laminating adhesive used was Adcote® 503E+co-reactant F (available commercially from Dow Chemical Company (Midland, Mich., United States of America)) prepared with 20-25% solids and at a batch size of 500 grams (Layer 2 from Table 2). A three layer structure was produced: EVA+gas generating composition (as described in Section III.B.)/laminating adhesive/PET sheet.

### Example 2

### Preparation of Film 2

Film 2 was prepared by cast coextrusion and adhesive lamination, as would be known to those of ordinary skill in the art.

### Example 3

### Preparation of Packages 1-5

5 packages were constructed using Film 1 as the lidding film and Film 2 as the bottom film. Particularly, Film 2 was used to construct a thermoformed pocket with the dimensions 3 inches×11 inches×0.5 inches (width×length×depth). A portion of Film 1 was then heat sealed to the formed pocket (2 seconds at 130° C.) to create a package with an enclosed headspace. Five packages (Packages 1-5) were constructed, each having a headspace of about 270 cm³.

### Example 4

### Triggering of Packages 1-5

Packages 1-5 were each placed into an enclosed plywood triggering box at various distances from triggering lamps and were triggered by exposing the packages to the light source for about 30 minutes to release chlorine dioxide into the package headspace. The triggering conditions are set forth below in Table 3.

TABLE 3
Triggering Conditions for Packages 1-5

| | | Lamp Distance | Triggering |
|---|---|---|---|
| Package | | from Package | Time |
| No. | Lamp Type | Lid (inches) | (min.) |
| 1 | One 500 W halogen bulb | 8 | 15 |
| 2 | Two 15 W fluorescent bulbs | 4 | 30 |
| 3 | One 15 W fluorescent bulb | 4 | 30 |
| 4 | One 500 W halogen bulb + | 4 | 1 (halogen) + |
| | one 15 W fluorescent bulb | | 10 |
| | | | (fluorescent) |
| 5 | One 500 W halogen bulb + | 4 | 1 (halogen) + |
| | Fifteen LED bulbs (to | | 10 |
| | produce light at 405 nm) | | (LED) |

### Example 5

### Generation of the Release Profiles for Packages 1-5

Release profiles were obtained using a mass spectrometer (Hewlett Packard, 5971A Mass Selective Detector, Agilent Technologies, Santa Clara, Calif., United States of America). One end of a deactivated fused silica capillary (5 m×0.1 mm) was attached to Package 1 to sample the package headspace in real time during triggering using the vacuum pump from the mass spectrometer. The other end of the capillary was positioned to feed into the ionization source of the quadrupole mass spectrometer. The sampled chlorine dioxide molecules were then ionized and separated according to the mass-to-charge ratio by the spectrometer, operating in selected ion monitoring (SIM) mode specifically for the mass-to-charge ratio of chlorine dioxide (67 amu, +1 charge). The total ion intensity of chlorine dioxide was then plotted versus time to construct a release profile. The process was repeated for Packages 2-5.

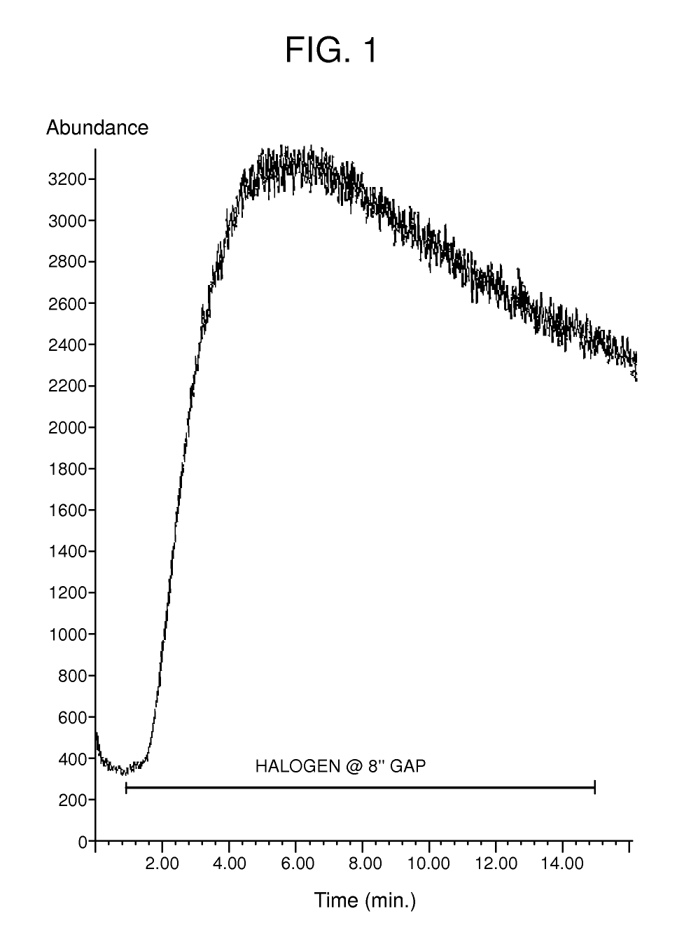

Release profiles of Packages 1-5 are shown in FIGS. 1-5. Due to mass spectrometer signal drift, the data was not used to accurately quantify the chlorine dioxide concentration inside Packages 1-5.

It was observed from FIG. 1 that the chlorine dioxide concentration within Package 1 rapidly increased to a maximum at about 5 minutes. After reaching the maximum concentration, chlorine dioxide continued to be produced from the gas generating composition (as described in Section III.B.), but began to decompose in the high intensity halogen light.

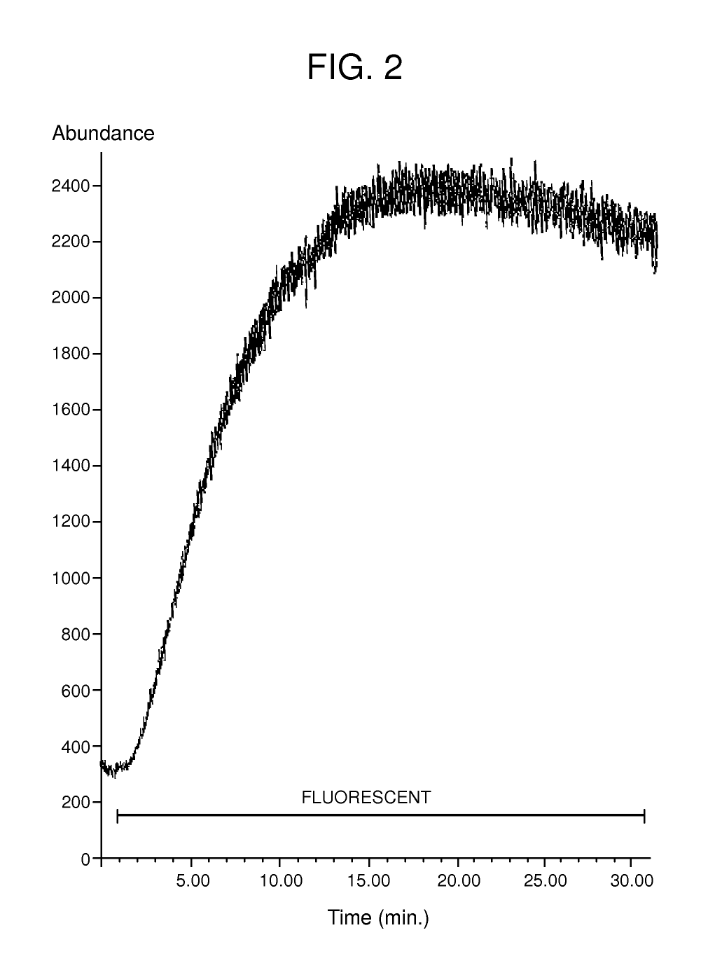

FIG. 2 illustrates that the maximum chlorine dioxide concentration for Package 2 was achieved at about 18 minutes. After reaching the maximum concentration, the chlorine dioxide concentration was much more stable when using the fluorescent lamp compared to the halogen lamp of Package 1.

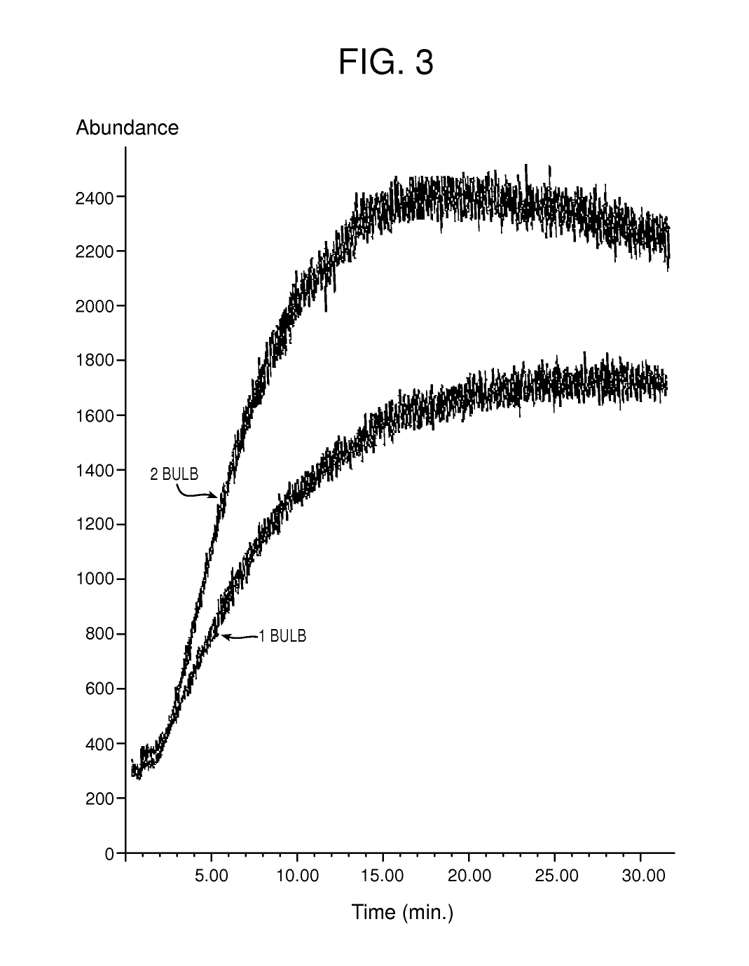

FIG. 3 shows the effect of intensity on the triggering reaction in Package 3 compared to Package 2. Particularly, FIG. 3 is a combined plot illustrating the chlorine dioxide release rate when using one fluorescent bulb (low release rate, Package 3) compared to two fluorescent bulbs (standard release rate, Package 2). FIG. 3 demonstrates that the intensity of light must be controlled and designed to correlate to the desired release rate of the gas.

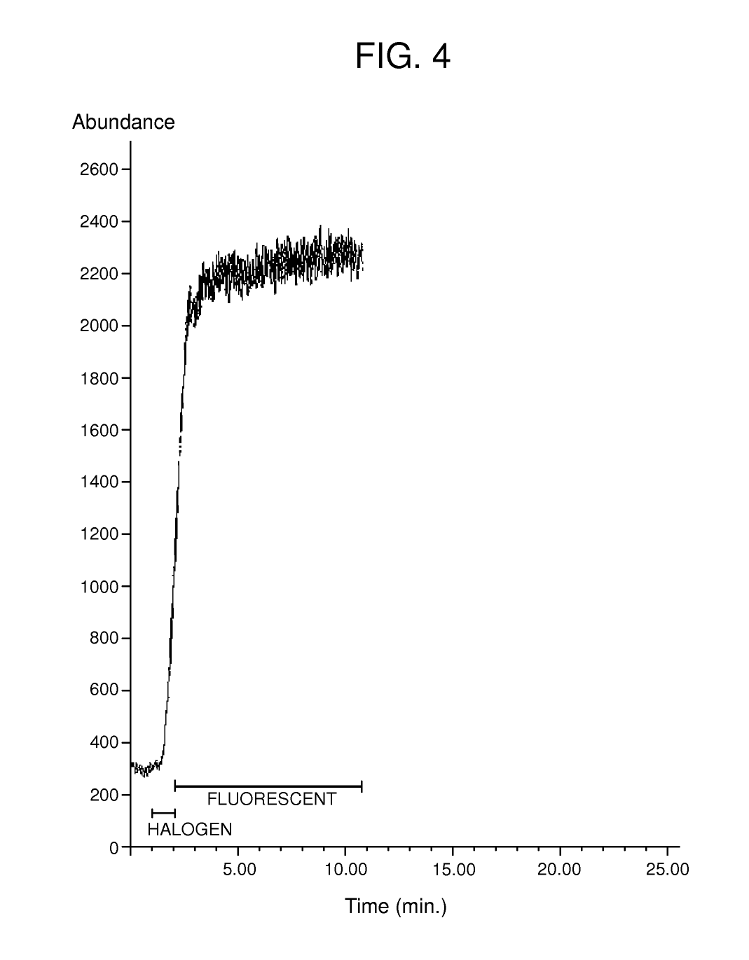

FIG. 4 illustrates an approximately optimum style curve for using the photoinitiator in a packaging format. Package 4 was triggered under a halogen lamp for 1 minute and then immediately placed under a fluorescent fixture for 10 minutes. The combination of lamps enabled both a rapid increase in chlorine dioxide concentration and a stable maintenance of the gas concentration level over time.

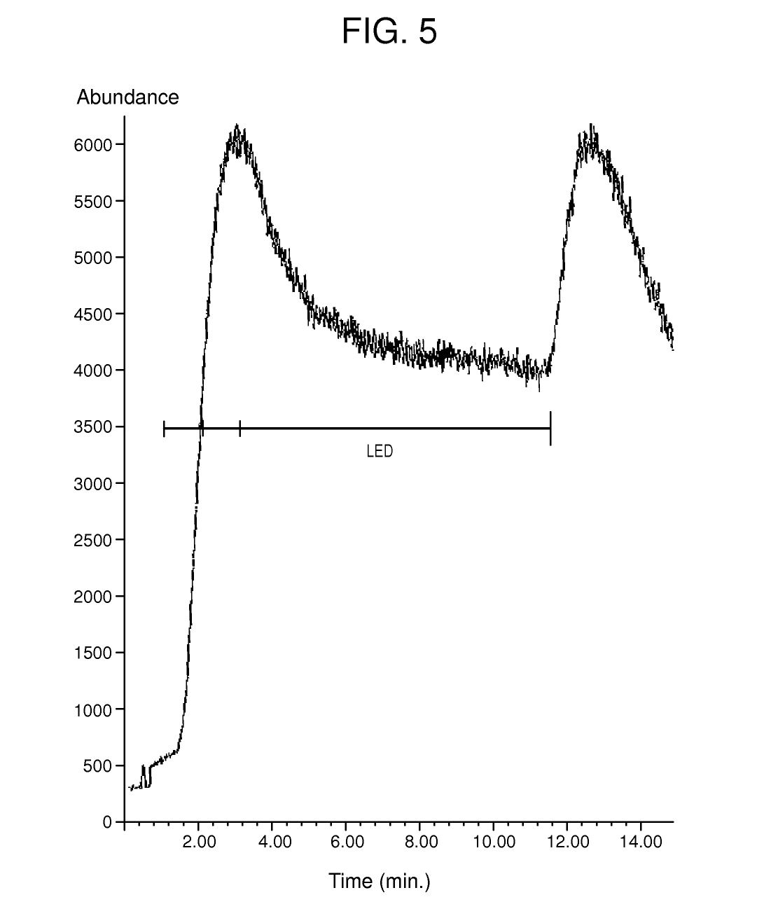

FIG. 5 illustrates a combination approach using an initial triggering of Package 5 under a halogen lamp for 1 minute, followed by immediately exposing the package to a bundle of LEDs for 10 minutes. It was noted that the release rate of Package 5 was inferior to Package 4 as a result of the limited intensity of light produced by the LEDs.

What is claimed is:

1. A system comprising:
a. a first area comprising at least one light source with a wavelength of about 350 to 1000 nm and a relative intensity of about 100;
b. a second area comprising at least one light source with a wavelength of about 385 to 750 nm and a relative intensity of 0.001 to 50; and
c. a package comprising:
i. a composition that generates a disinfecting gas upon exposure to said first and second light sources; and
ii. an object to be disinfected.

2. The system of claim 1, wherein said package comprises at least one polymeric film and said composition is incorporated within at least one layer of said polymeric film.

3. The system of claim 1, wherein said first and second areas are in a spiral configuration.

4. The system of claim 1, wherein said composition comprises an energy-activated catalyst and anions capable of being oxidized by the activated catalyst or a subsequent reaction product to generate a gas.

5. The system of claim 1, wherein said disinfecting gas is selected from the group consisting of: chlorine dioxide, ethylene oxide, sulfur dioxide, hydrogen sulfide, hydrocyanic acid, nitrogen dioxide, nitric oxide, nitrous oxide, carbon dioxide, dichlorine monoxide, vaporous hydrogen peroxide, chlorine, ozone, or combinations thereof.

6. The system of claim 1, wherein said first light source is selected from the group consisting of: sunlight, fluorescent, ultraviolet, tungsten halogen, LED, xenon, metal halide, high pressure mercury vapor, arc lamp, sodium vapor lamp, high pressure sodium lamp, induction lamp, electroluminescent, OLED, and combinations thereof.

7. The system of claim 1, wherein said second light source is selected from the group consisting of: sunlight, fluorescent, ultraviolet, tungsten halogen, LED, xenon, metal halide, high pressure mercury vapor, arc lamp, sodium vapor lamp, high pressure sodium lamp, induction lamp, electroluminescent, OLED, and combinations thereof.

8. The system of claim 1, wherein said object is a medical device.

9. A method of controlling the generation of a disinfecting gas within a package interior, said method comprising:
a. providing the package of claim 1 in an unactivated state;
b. introducing the package to the system of claim 1;
wherein the system exposes the package to at least one light source with a wavelength of 350 to 1000 nm and a relative intensity of about 100 for a period of about 1 to 3 minutes followed by immediate exposure to at least one light source with a wavelength of 385 to 750 nm and a relative intensity of about 0.001 to 50 for a period of about 10 to 30 minutes; and
wherein the package exhibits a controlled generation of the gas.

10. The method of claim 9, wherein said package comprises at least one polymeric film and said composition is incorporated within at least one layer of said polymeric film.

11. The method of claim 9, wherein said composition comprises an energy-activated catalyst and anions capable of being oxidized by the activated catalyst or a subsequent reaction product to generate a gas.

12. The method of claim 9, wherein said disinfecting gas is selected from the group consisting of: chlorine dioxide, ethylene oxide, sulfur dioxide, hydrogen sulfide, hydrocyanic acid, nitrogen dioxide, nitric oxide, nitrous oxide, carbon dioxide, vaporous hydrogen peroxide, dichlorine monoxide, chlorine, ozone, or combinations thereof

13. A method of providing sustained generation of a disinfecting gas within a package, said method comprising:
a. providing the package of claim 1 in an unactivated state;
b. introducing the package to the system of claim 1;
wherein the system exposes the package to at least one light source with a wavelength of 350 to 1000 nm and a relative intensity of about 100 for a period of about 1 to 3 minutes followed by immediate exposure to at least one light source with a wavelength of 400 to 725 nm and a relative intensity of about 0.001 to 50 for a period of about 10 to 30 minutes; and
wherein the package exhibits sustained generation of the gas at a level of 400 ppmv or less.

14. The method of claim 13, wherein said package comprises at least one polymeric film and said composition is incorporated within at least one layer of said polymeric film.

15. The method of claim 13 wherein said composition comprises an energy-activated catalyst and anions capable of being oxidized by the activated catalyst or a subsequent reaction product to generate a gas.

16. The method of claim 13, wherein said disinfecting gas is selected from the group consisting of: chlorine dioxide, ethylene oxide, sulfur dioxide, hydrogen sulfide, hydrocyanic acid, nitrogen dioxide, vaporous hydrogen peroxide, nitric oxide, nitrous oxide, carbon dioxide, dichlorine monoxide, chlorine, ozone, or combinations thereof.

17. A system comprising:
a. an area comprising at least one light source comprising:
i. a first set of lenses, mirrors, filters, or combinations thereof that provide a wavelength of about 350 to 1000 nm and a relative intensity of about 100; and
ii. a second set of lenses, mirrors, or combinations thereof that provide a wavelength of about 385 to 750 nm and a relative intensity of 0.001 to 50; and
b. a package comprising:
i. a composition that generates a disinfecting gas upon exposure to said light source; and
ii. an object to be disinfected.

18. A method of controlling the generation of a disinfecting gas within a package interior, said method comprising:
a. providing the package of claim 17 in an unactivated state;
b. introducing the package to the system of claim 17;
wherein the system exposes the package to a wavelength of 350 to 1000 nm and a relative intensity of about 100 for a period of about 1 to 3 minutes followed by immediate exposure to a wavelength of 385 to 750 nm and a relative intensity of about 0.001 to 50 for a period of about 10 to 30 minutes; and
wherein the package exhibits a controlled generation of the gas.

19. A method of providing sustained generation of a disinfecting gas within a package, said method comprising:
a. providing the package of claim 17 in an unactivated state;
b. introducing the package to the system of claim 17;
wherein the system exposes the package to a wavelength of 350 to 1000 nm and a relative intensity of about 100 for a period of about 1 to 3 minutes followed by immediate exposure to a wavelength of 400 to 725 nm and a relative intensity of about 0.001 to 50 for a period of about 10 to 30 minutes; and
wherein the package exhibits sustained generation of the gas at a level of 400 ppmv or less.
